(19) United States
(12) Patent Application Publication
(10) Pub. No.: US 2012/0243663 A1
(43) Pub. Date: Sep. 27, 2012

(54) X-RAY IMAGING SYSTEM, METHOD OF CONTROLLING X-RAY IMAGING SYSTEM, INFORMATION PROCESSING APPARATUS, AND X-RAY IMAGING APPARATUS

(75) Inventor: Yuichi Nishii, Kawasaki-shi (JP)

(73) Assignee: CANON KABUSHIKI KAISHA, Tokyo (JP)

(21) Appl. No.: 13/403,070

(22) Filed: Feb. 23, 2012

(30) Foreign Application Priority Data
Mar. 23, 2011 (JP) 2011-064828

(51) Int. Cl. H05G 1/64

(52) U.S. Cl. 378/98

(57) ABSTRACT

An X-ray imaging system comprising an X-ray imaging apparatus which captures an X-ray image and an information processing apparatus which receives and composes the X-ray image, the X-ray imaging apparatus comprising a transmission unit configured to segment a X-ray image into images and sequentially transmit the images, and the information processing apparatus comprising a reception/composition unit configured to sequentially receive and compose the X-ray images, a determination unit configured to determine, every time the X-ray images are sequentially composed, whether a resolution of the composed X-ray image is not less than a predetermined value, and a display control unit configured to perform control to display the composed X-ray image on a display unit, when the determination unit determines that the resolution is not less than the predetermined value.

### BACKGROUND OF THE INVENTION

1. Field of the Invention

The present invention relates to an X-ray imaging system, a method of controlling the X-ray imaging system, an information processing apparatus, and an X-ray imaging apparatus and, more particularly, to an X-ray imaging system which reduces display delays by executing segmented transfer/reception of image data and displaying an image in accordance with the resolution even during reception, a method of controlling the X-ray imaging system, an information processing apparatus, and an X-ray imaging apparatus.

2. Description of the Related Art

There have been growing needs to improve efficiency and speed up examination by digitalizing and storing/transmitting medical image information of patients which is generated in a hospital. Under this circumstance, in the field of general X-ray imaging, a digital system which outputs digital data using an X-ray detector such as an FPD (Flat Panel Detector) in place of a conventionally used screen/film system has been increasingly used.

Recently, some digital systems like that described above use wireless transmission to improve portability. Although the wireless data rate has increased, the transmission rate is lower than wired. In addition, the recent increases in the resolution of images tend to increase the time required to display an image after it is captured, that is, to cause a display delay.

In consideration of such tendency, Japanese Patent Laid-Open No. 2006-26083 discloses a method of displaying a sampled reduced image in advance to speed up display processing. There is also disclosed a method of generating a sampled image which allows to execute analysis for grid stripe suppression processing.

The conventional system, however, is configured to display a sampled image only for the purpose of checking whether imaging has been properly performed. That is, this system has a problem that it does not speed up displaying of a high-resolution image to be displayed afterward.

In consideration of the above problem, the present invention provides a technique of displaying a high-resolution image at high speed.

### SUMMARY OF THE INVENTION

According to one aspect of the present invention, there is provided an X-ray imaging system comprising an X-ray imaging apparatus which captures an X-ray image and an information processing apparatus which receives and composes the X-ray image which has been segmented and transmitted, the X-ray imaging apparatus comprising a transmission unit configured to segment a captured X-ray image into images and sequentially transmit the images, and the information processing apparatus comprising a reception/composition unit configured to sequentially receive and compose the segmented and transmitted X-ray images, a determination unit configured to determine, every time the X-ray images received by the reception/composition unit are sequentially composed, whether a resolution of the composed X-ray image is not less than a predetermined value, and a display control unit configured to perform control to display the composed X-ray image on a display unit, when the determination unit determines that a resolution of the composed X-ray image is not less than the predetermined value.

According to one aspect of the present invention, there is provided a method of controlling an X-ray imaging system comprising an X-ray imaging apparatus which captures an X-ray image and an information processing apparatus which receives and composes the X-ray image which has been segmented and transmitted, the method comprising: segmenting, by a transmission unit of the X-ray imaging apparatus, a captured X-ray image into images and sequentially transmit the images; receiving and composing, by a reception/composition unit of the information processing apparatus, sequentially the segmented and transmitted X-ray images; determining, by a determination unit of the information processing apparatus, every time the X-ray images received in the receiving/composing are sequentially composed, whether a resolution of the composed X-ray image is not less than a predetermined value; and performing, by a display control unit of the information processing apparatus, control to display the composed X-ray image on a display unit, when it is determined in the determining that a resolution of the composed X-ray image is not less than the predetermined value.

According to one aspect of the present invention, there is provided an information processing apparatus which receives and composes an X-ray image which has been segmented and sequentially transmitted from an X-ray imaging apparatus, the information processing apparatus comprising: a reception/composition unit configured to sequentially receive and compose the segmented and transmitted X-ray images; a determination unit configured to determine, every time the X-ray images received by the reception/composition unit are sequentially composed, whether a resolution of the composed X-ray image is not less than a predetermined value; and a display control unit configured to perform control to display the composed X-ray image on a display unit, when the determination unit determines that a resolution of the composed X-ray image is not less than the predetermined value.

According to one aspect of the present invention, there is provided an X-ray imaging apparatus which captures an X-ray image, the apparatus comprising: a region segmentation unit configured to segment the X-ray image into regions each including N pixels×N pixels; a selection unit configured to select N pixels for the each region without overlapping; and a pixel transmission unit configured to transmit N pixels selected for the each region, wherein the selection unit selects N pixels different from the N pixels for the each region without overlapping when performing next transmission.

Further features of the present invention will be apparent from the following description of exemplary embodiments with reference to the attached drawings.

### BRIEF DESCRIPTION OF THE DRAWINGS

FIG. 1 is a block diagram showing the schematic arrangement of an X-ray imaging system;

FIG. 2 is a flowchart showing a procedure for segmented transmission processing performed by an X-ray image segmented transmission unit;

FIG. 3 is a flowchart showing a procedure for the processing (S205) of selecting N pixels in a map of N pixels×N pixels;

FIG. 4 is a flowchart showing a procedure for the processing of receiving, composing, and displaying images using an image reception/composition unit;

FIG. 5 is a view showing the state of a map when an image of 16 pixels×24 pixels is segmented into eight segments;

FIG. 6 is a view showing a selection method A for pixels in an 8×8 map in the first segmentation;

FIG. 7 is a view showing the selection method A for pixels in the 8×8 map in the second segmentation;

FIG. 8 is a view showing a selection method B for pixels in an 8×8 map in the first segmentation;

FIG. 9 is a view showing a selection method C for pixels in an 8×8 map in the first segmentation;

FIG. 10 is a view showing the selection method B for pixels in the 8×8 map in the second segmentation;

FIG. 11 is a view showing the selection method B for pixels in the 8×8 map in the third segmentation;

FIG. 12 is a view showing the selection method B for pixels in the 8×8 map in the fourth segmentation;

FIG. 13 is a view showing the selection method B for pixels in the 8×8 map in the eighth segmentation;

FIG. 14 is a view showing an example of a reconstruction result in the first segmentation in eight-segmentation processing;

FIG. 15 is a view showing an example of a reconstruction result in the second segmentation of eight-segmentation processing;

FIG. 16 is a view showing an example of a reconstruction result in the third segmentation of eight-segmentation processing; and

FIG. 17 is a view showing an example of a reconstruction result in the fourth segmentation of eight-segmentation processing.

### DESCRIPTION OF THE EMBODIMENTS

An exemplary embodiment(s) of the present invention will now be described in detail with reference to the drawings. It should be noted that the relative arrangement of the components, the numerical expressions and numerical values set forth in these embodiments do not limit the scope of the present invention unless it is specifically stated otherwise.

### First Embodiment

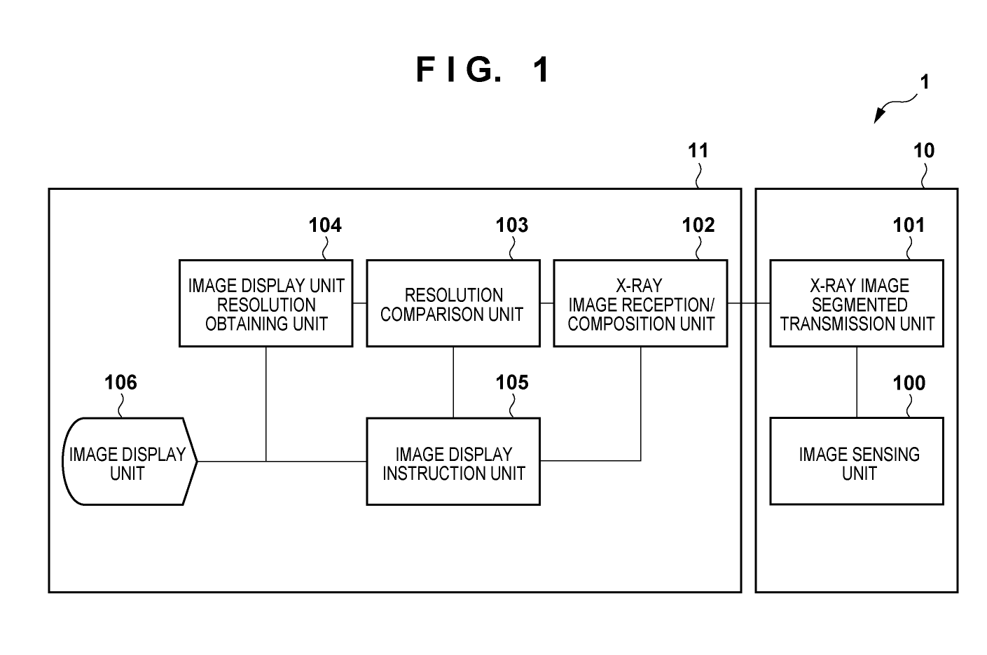

The first embodiment will be described below with reference to the accompanying drawings. FIG. 1 is a block diagram showing the arrangement of an X-ray imaging system 1 according to this embodiment.

The X-ray imaging system 1 includes an X-ray imaging apparatus 10 and an information processing apparatus 11. The X-ray imaging apparatus 10 includes an image sensing unit 100 and an X-ray image segmented transmission unit 101. The information processing apparatus 11 includes an X-ray image reception/composition unit 102, a resolution comparison unit 103, an image display unit resolution obtaining unit 104, an image display instruction unit 105, and an image display unit 106.

A CPU (not shown) controls each processing unit. The CPU controls each processing unit by reading out and executing the control programs stored in a storage unit (not shown).

The image sensing unit 100 captures an X-ray image, and includes, for example, an X-ray emission apparatus and an X-ray detection apparatus. Upon execution of X-ray imaging, the X-ray image segmented transmission unit 101 segments an X-ray image and sequentially transmits the segmented image data. When sequentially receiving the segmented image data, the X-ray image reception/composition unit 102 sequentially composes the received segmented image data. The resolution comparison unit 103 compares the resolution of the image composed by the X-ray image reception/composition unit 102 with the resolution of the screen obtained by the image display unit resolution obtaining unit 104. The image display unit resolution obtaining unit 104 obtains the resolution of the image display instruction unit 105. Based on the comparison result obtained by the resolution comparison unit 103, the image display instruction unit 105 instructs the image display unit 106 to display an image. The image display unit 106 displays an X-ray image on the screen.

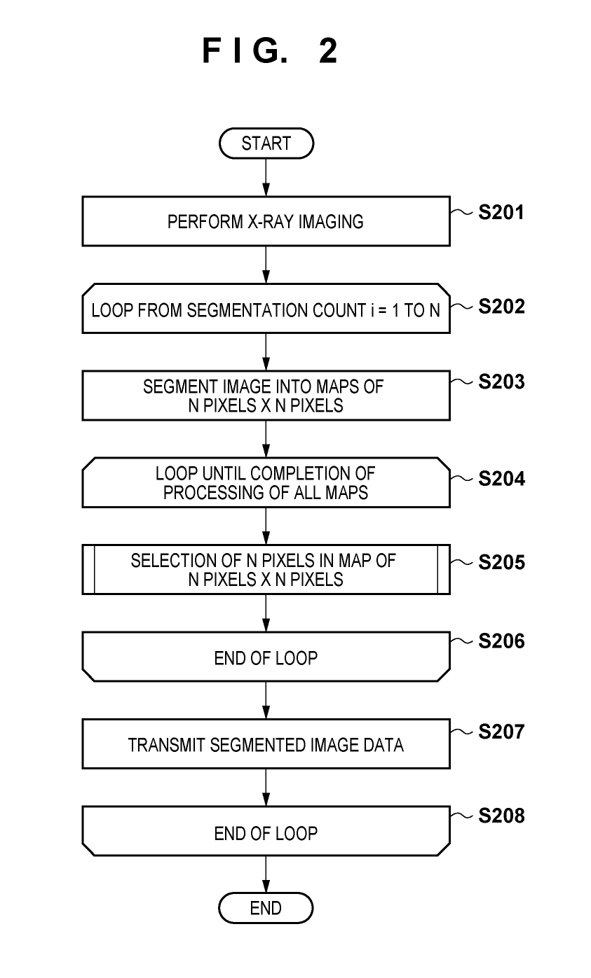

A processing procedure executed by the X-ray image segmented transmission unit 101 of the X-ray imaging apparatus 10 will be described next with reference to the flowchart of FIG. 2.

In step S201, the image sensing unit 100 executes X-ray imaging. In steps S202 to S208, when segmenting a captured image into N segments, the X-ray image segmented transmission unit 101 executes the loop from segmentation count i=1 to N.

In step S203, for N segmentation, the X-ray image segmented transmission unit 101 segments the captured image into maps each constituted by N pixels×N pixels. In steps S204 to S206, the X-ray image segmented transmission unit 101 executes the loop until processing all the maps.

In step S205, the X-ray image segmented transmission unit 101 selects N pixels in a map of N pixels×N pixels, obtained by segmentation, based on a predetermined rule. The predetermined rule will be described in detail later.

In step S207, upon completion of the selection processing in steps S204 to S206, the X-ray image segmented transmission unit 101 transmits the segmented image (pixel transmission processing). Subsequently, the X-ray image segmented transmission unit 101 repeats the above operation from segmentation count i=1 to N.

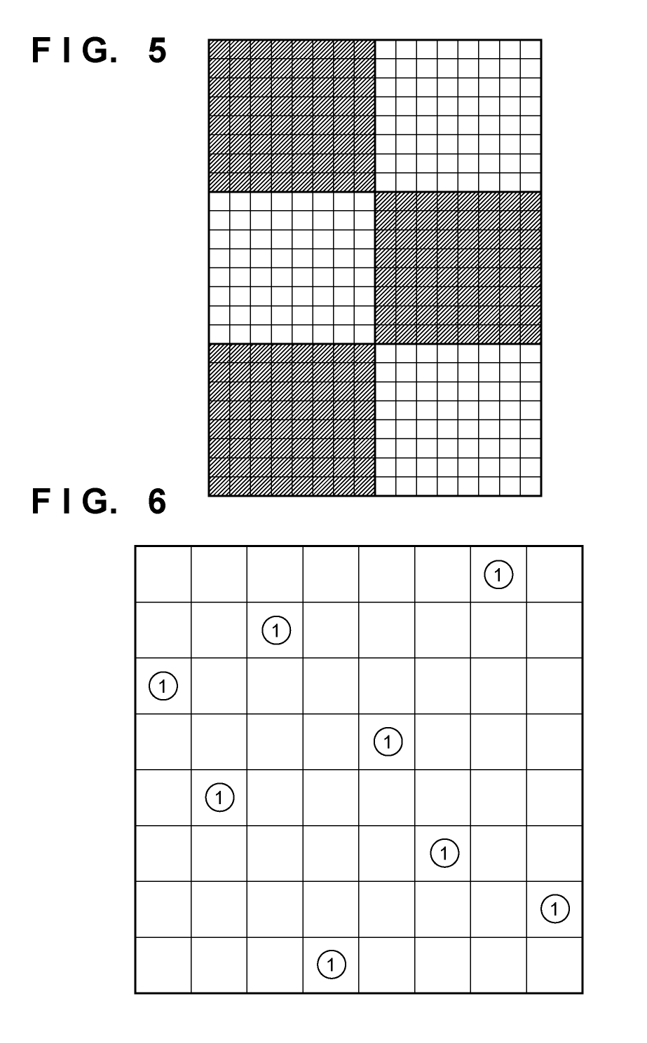

The processing procedure described with reference to FIG. 2 will be described concretely. FIG. 5 shows a case in which an image with a size of 16×24, that is, 16 pixels in the horizontal direction×24 pixels in the vertical direction, is segmented into eight segments with N=8. As shown in FIG. 5, the image is segmented into six maps each constituted by 8 pixels×8 pixels. The X-ray image segmented transmission unit 101 selects eight pixels from an 8×8 map based on a predetermined rule, and transmits the image. The X-ray image segmented transmission unit 101 repeats this operation eight times.

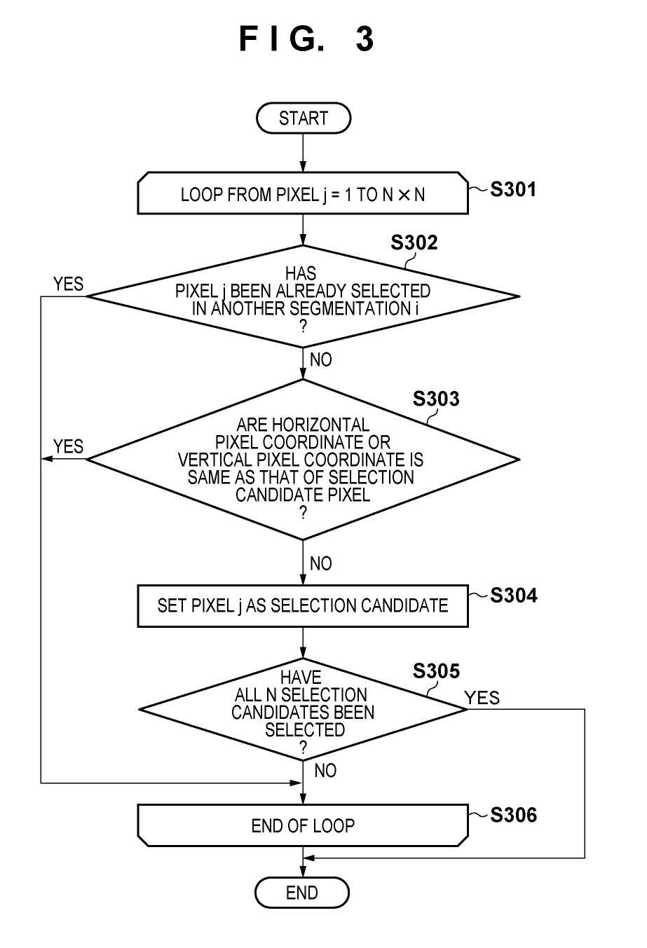

A method of selecting pixels from a map of N pixels×N pixels based on a predetermined rule in step S205 will be described next with reference to the flowchart of FIG. 3.

In step S301, the X-ray image segmented transmission unit 101 selects an arbitrary pixel j by scanning the pixels in the map of N pixels×N pixels (8 pixels×8 pixels). In step S302, the X-ray image segmented transmission unit 101 determines whether the pixel j has already been selected in another segmentation i. If the X-ray image segmented transmission unit 101 determines that the pixel j has already been selected (YES in step S302), the process shifts to the next pixel. If the X-ray image segmented transmission unit 101 determines that the pixel j has not been selected (NO in step S302), the process advances to step S303.

In step S303, the X-ray image segmented transmission unit 101 determines whether the currently selected pixel j has the same horizontal pixel coordinate or vertical pixel coordinate as that of a selection candidate pixel. If the X-ray image segmented transmission unit 101 determines that the pixel j has the same horizontal pixel coordinate or vertical pixel coordinate as that of the selection candidate pixel, that is, at least one of the coordinates coincides with that of the selection candidate pixel (YES in step S303), the process shifts to the next pixel. If the X-ray image segmented transmission unit 101 determines that neither the horizontal pixel coordinate nor the vertical pixel coordinate of the pixel j coincides with that of the selection candidate pixel (NO in step S303), the process advances to step S304.

In step S304, the X-ray image segmented transmission unit 101 sets the selected pixel j as a selection candidate. In step S305, the X-ray image segmented transmission unit 101 determines whether N pixels are all selected as selection candidates. If the X-ray image segmented transmission unit 101 determines that N pixels are all selected as selection candidates (YES in step S305), the process advances to step S306. If the X-ray image segmented transmission unit 101 determines that N pixels are not all selected as selection candidates (NO in step S305), the process shifts to the next pixel.

In step S306, if the execution of the loop from segmentation count i=1 to N is complete, the X-ray image segmented transmission unit 101 terminates the processing.

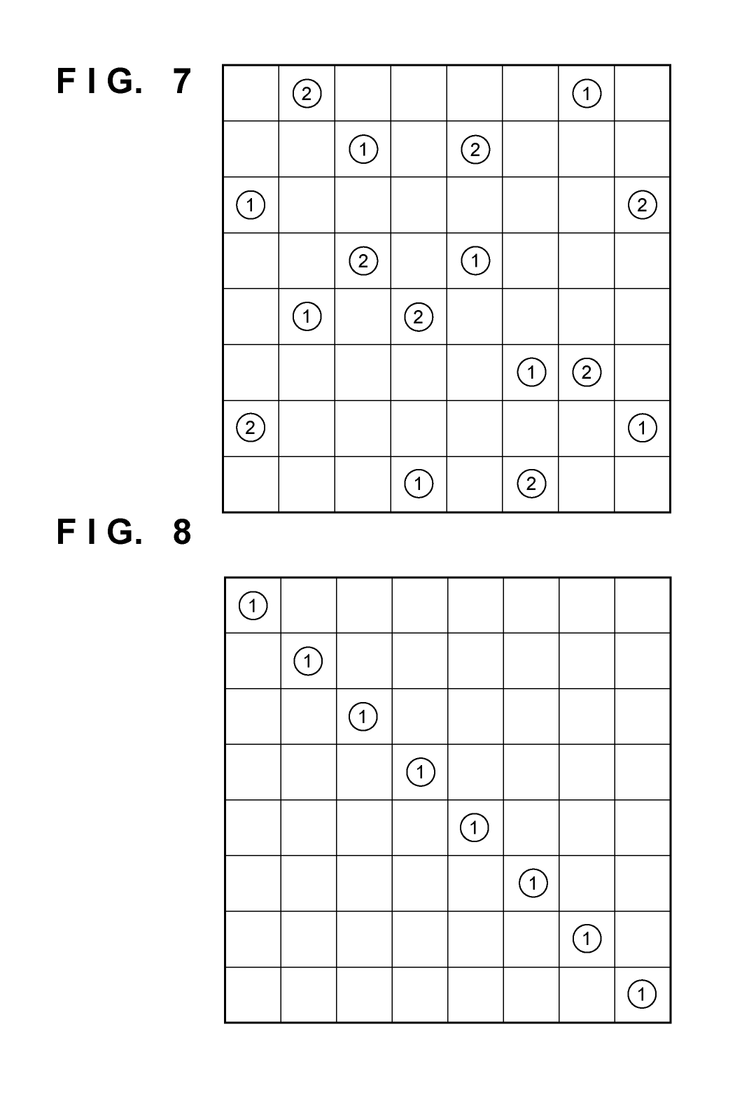

The processing based on the above flowchart will be concretely described. When an image is segmented into 8 pixels×8 pixels as shown in FIG. 6, the X-ray image segmented transmission unit 101 selects pixels in the first segmentation such that neither horizontal pixel coordinates nor vertical pixel coordinates overlap. As shown in FIG. 7, in the second segmentation, the X-ray image segmented transmission unit 101 also selects pixels, other than the pixels selected in the first segmentation, such that neither horizontal pixel coordinates nor vertical pixel coordinates overlap. The X-ray image segmented transmission unit 101 selects N pixels from each region constituted by N pixels×N pixels such that the horizontal coordinates and vertical coordinates of the N pixels do not overlap on orthogonal coordinates with a predetermined pixel of the region being an origin point.

The purpose of selecting pixels while preventing horizontal pixel coordinates and vertical pixel coordinates from overlapping in this manner is to suppress the formation of grid stripes and to segment the image so as to allow to obtain all pieces of vertical and horizontal line information.

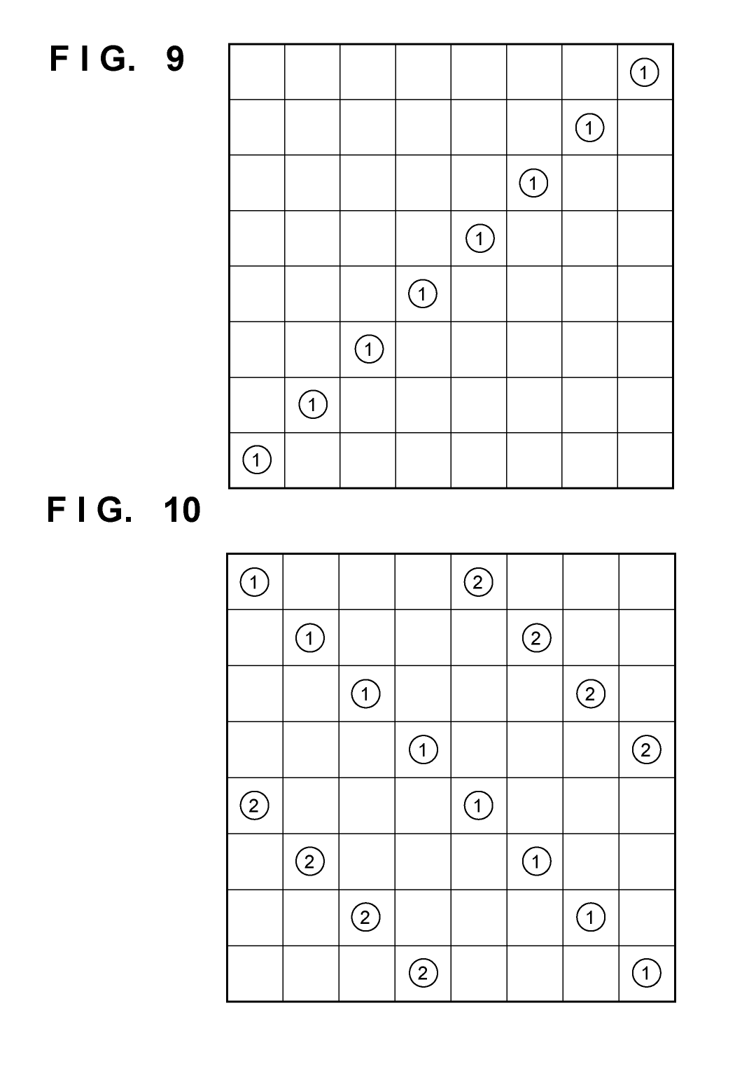

Although the method of randomly obtaining pixels while preventing the horizontal pixel coordinates and the vertical pixel coordinates from overlapping has been described above with reference to FIGS. 6 and 7, a more typical method is to select pixels arranged in diagonal directions on the map shown in FIGS. 8 and 9 (that is, to select pixels arranged along a vector in either a 45° direction or a −45° direction, when the horizontal pixel direction is defined as 0° and the vertical pixel direction is defined as 90°, with the upper left coordinates of the image being the origin point). An advantage of this method is to allow to continuously obtain line information constituted by consecutive pixels, and hence to allow to obtain more accurate information.

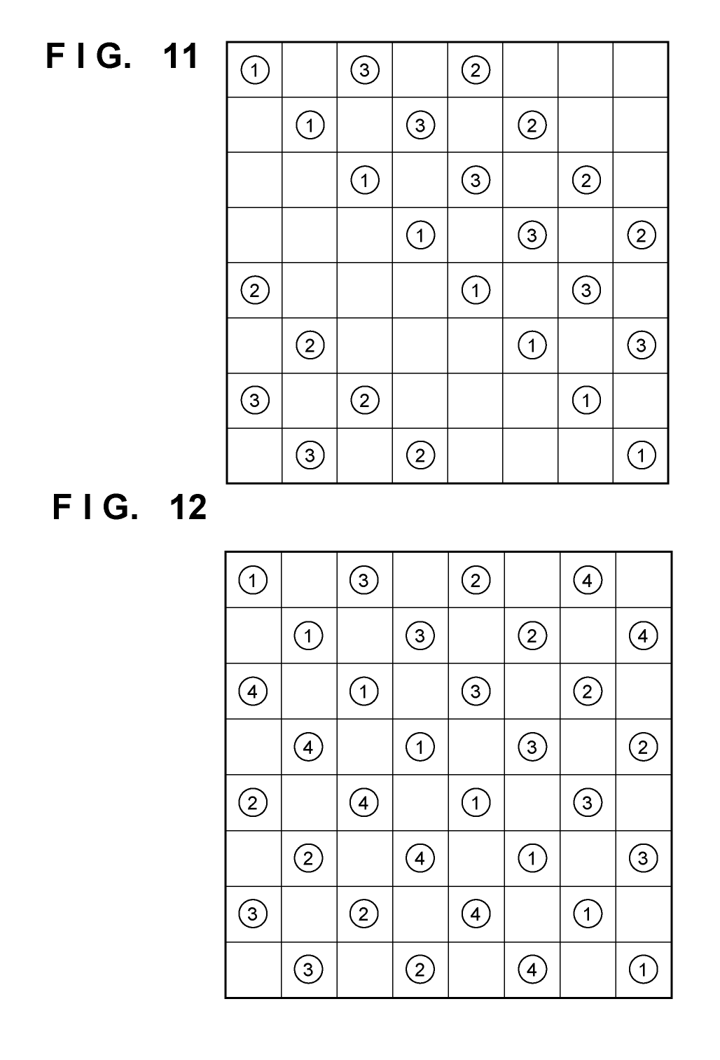

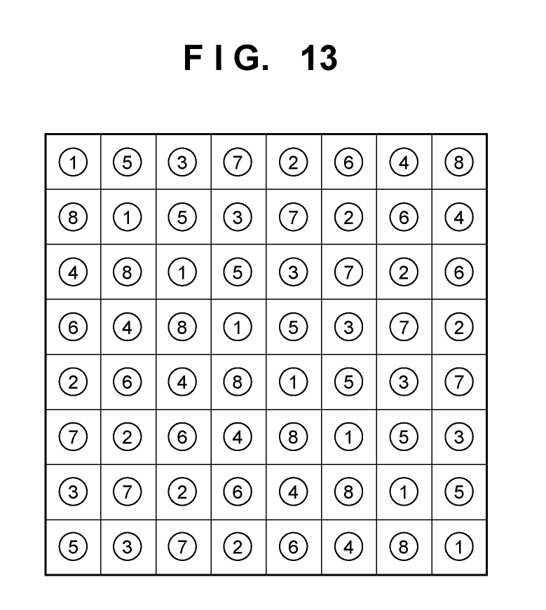

In the case shown in FIG. 8, in the second segmentation, the X-ray image segmented transmission unit 101 selects pixels so as to interpolate pixels with respect to the first segmentation as shown in FIG. 10, and selects pixels in the third and fourth segmentations so as to interpolate pixels between the first segmentation and the second segmentation, as shown in FIGS. 11 and 12. FIG. 13 shows the result obtained by segmenting a map of 8 pixels×8 pixels into eight segments in this manner.

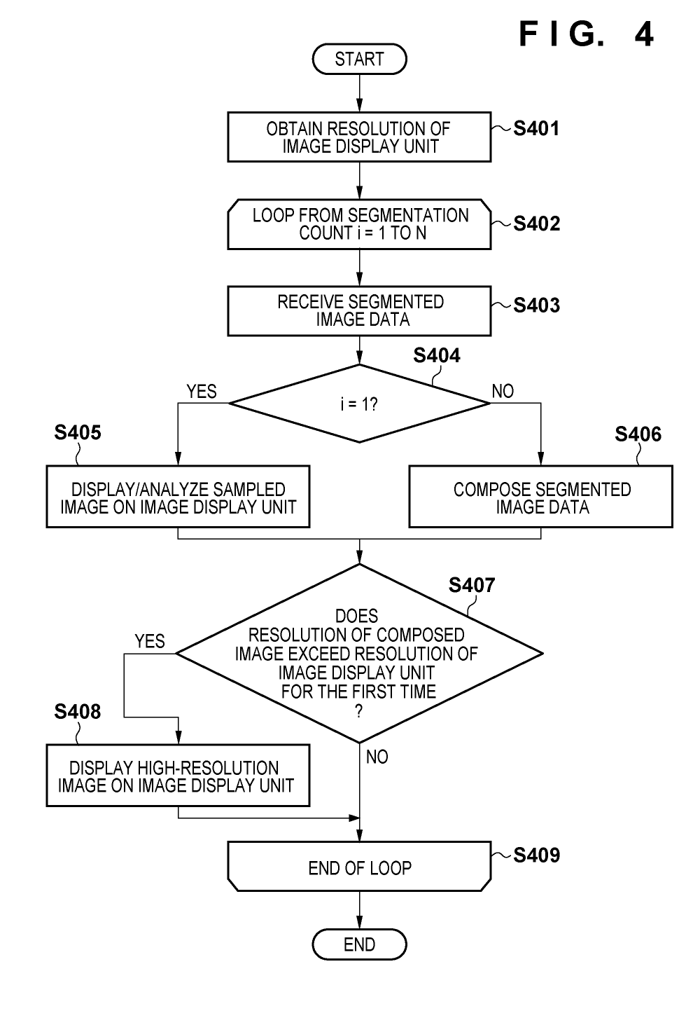

A processing procedure from the reception of an image to screen display will be described next with reference to the flowchart of FIG. 4.

In step S401, the image display unit resolution obtaining unit 104 obtains the resolution of the image display unit 106. In steps S402 to S409, when X-ray image segmented transmission unit 101 has transmitted an image upon segmenting it into N segments, the information processing apparatus executes the loop from segmentation count i=1 to N.

In step S403, the X-ray image reception/composition unit 102 receives the image transmitted from the X-ray image segmented transmission unit 101 upon segmentation. In step S404, the X-ray image reception/composition unit 102 determines whether segmentation count i=1. If the X-ray image reception/composition unit 102 determines that segmentation count i=1 (YES in step S404), the process advances to step S405. If the X-ray image reception/composition unit 102 determines that segmentation count i≠1 (NO in step S404), the process advances to step S406.

In step S405, the X-ray image reception/composition unit 102 controls the image display unit 106 to display the image obtained in the first segmentation as a sampled image to check whether proper imaging has been performed, and executes analysis on grid stripes and the like.

In step S406, in the second segmentation, the X-ray image reception/composition unit 102 composes the segmented images obtained in the first and second segmentations. In the third segmentation, the X-ray image reception/composition unit 102 composes the segmented images obtained in the first, second, and third segmentations. In the fourth segmentation, the X-ray image reception/composition unit 102 composes the segmented images obtained in the first, second, third, and fourth segmentations. Subsequently, the X-ray image reception/composition unit 102 composes segmented imaged in the same manner.

In step S407, the resolution comparison unit 103 compares the resolution of this composed image with the resolution of the image display unit 106 which is obtained by the image display unit resolution obtaining unit 104 in step S401 to determine whether the resolution of the composed image exceeds the resolution of the image display unit 106 for the first time. In this case, the resolution of the image display unit 106 is used as a criterion. However, this apparatus may be configured to compare the resolution of a composed image with a predetermined value as a criterion. If the resolution comparison unit 103 determines that the resolution of the composed image becomes equal to or more than the resolution (or a predetermined value) of the image display unit 106 for the first time (YES in step S407), the process advances to step S408. If the resolution of the composed image has not become equal to or more than the resolution (or the predetermined value) of the image display unit 106 for the first time (NO in step S407), the process advances to step S409.

In step S408, the image display unit 106 displays the composed image as a high-resolution image in accordance with an instruction from the image display instruction unit 105. In step S409, upon receiving all the segmented images by continuously performing segmented reception, the apparatus finishes the loop from segmentation count i=1 to N, and terminates the processing.

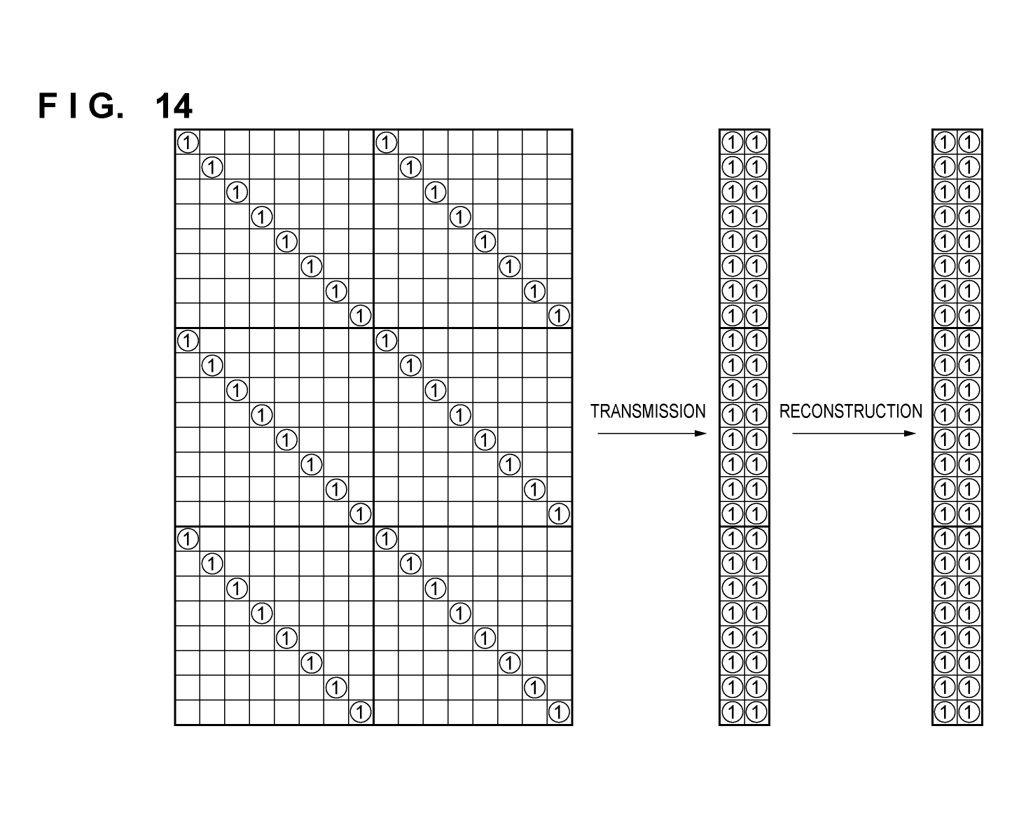

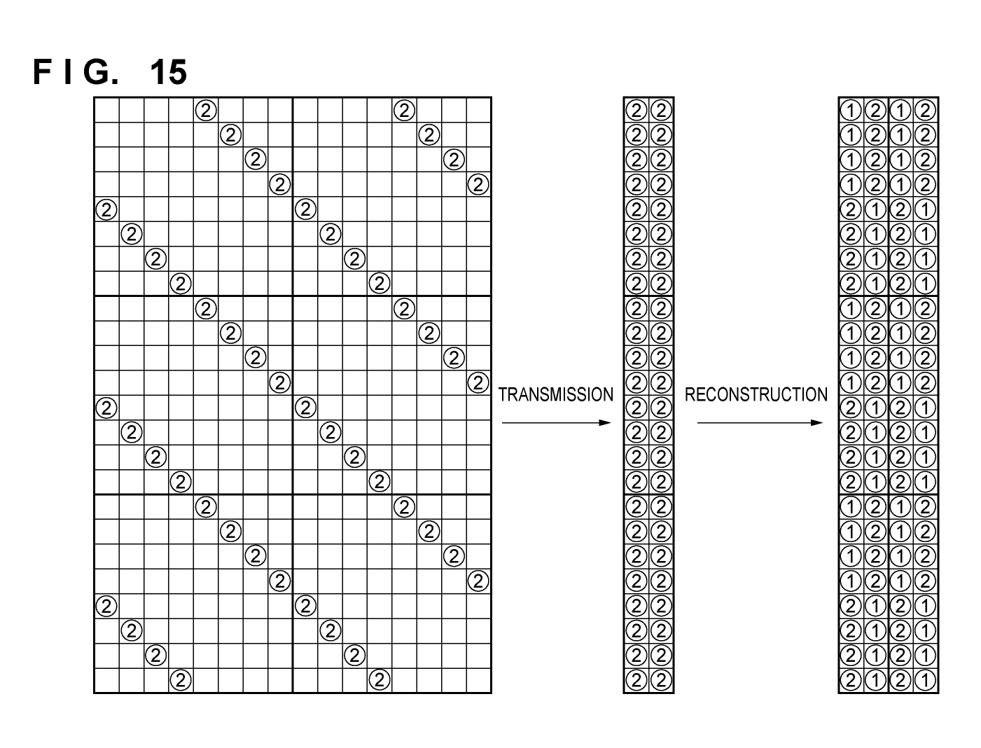

For example, when performing eight-segmentation processing, the X-ray image segmented transmission unit 101 executes selection like that shown in FIG. 14 in the first segmentation, and transmits the resultant image data. The X-ray image reception/composition unit 102 reconstructs the corresponding image.

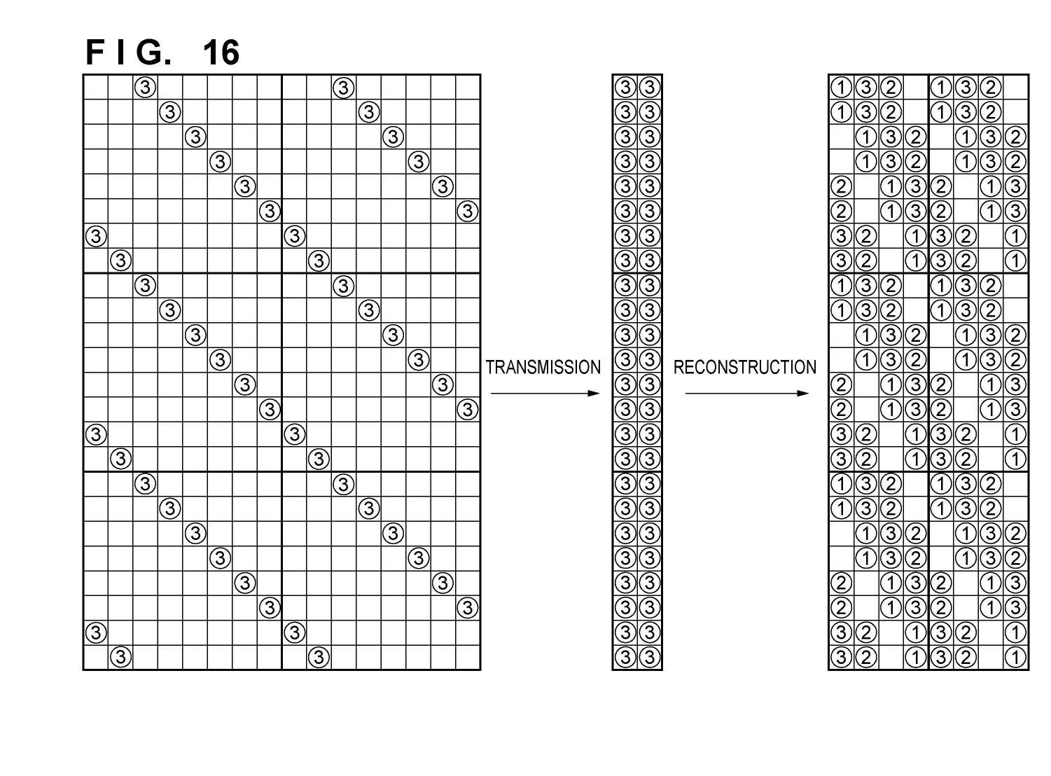

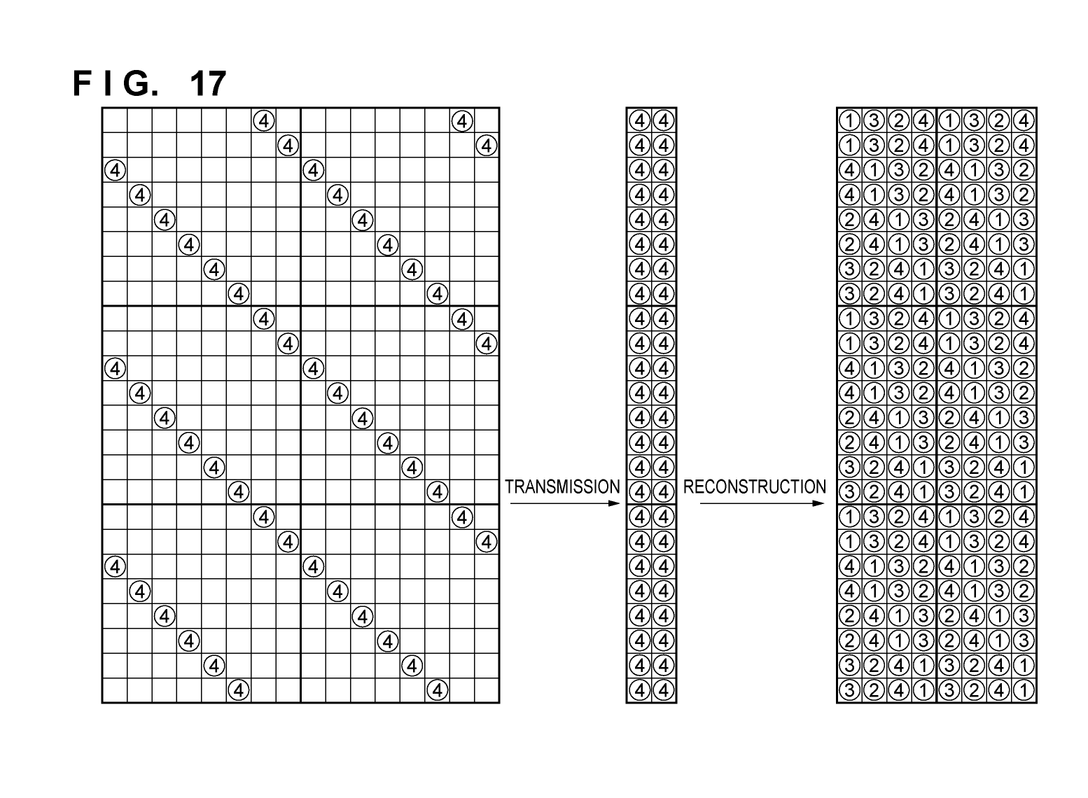

In the second segmentation, the images obtained in the first and second segmentations are composed and reconstructed as an image like that shown in FIG. 15. In the third segmentation, the images obtained in the first, second, and third segmentations are composed and reconstructed as an image like that shown in FIG. 16. In the fourth segmentation, the images obtained in the first, second, third, and fourth segmentations are composed and reconstructed as an image like that shown in FIG. 17.

X-ray detection apparatuses exceeding about 3000 pixels×3000 pixels have been developed. On the other hand, the screen resolution of the image display unit 106 is about 1600 pixels×1200 pixels, and the display area for images often has a resolution of 1200 pixels×1200 pixels at maximum.

In such a case, when performing eight-segmentation processing, for example, the apparatus can obtain an image with a resolution of 1500 pixels×3000 pixels in the fourth segmentation. That is, the apparatus has an image allowing high-resolution display even at this time when the apparatus has not received all the image data. Executing high-resolution display at this time can reduce display delays, thus speeding up the display processing.

According to the present invention, it is possible to display high-resolution images at high speed.

### Other Embodiments

Aspects of the present invention can also be realized by a computer of a system or apparatus (or devices such as a CPU or MPU) that reads out and executes a program recorded on a memory device to perform the functions of the above-described embodiment(s), and by a method, the steps of which are performed by a computer of a system or apparatus by, for example, reading out and executing a program recorded on a memory device to perform the functions of the above-described embodiment(s). For this purpose, the program is provided to the computer for example via a network or from a recording medium of various types serving as the memory device (for example, computer-readable storage medium).

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2011-064828 filed on Mar. 23, 2011, which is hereby incorporated by reference herein in its entirety.

What is claimed is:

1. An X-ray imaging system comprising an X-ray imaging apparatus which captures an X-ray image and an information processing apparatus which receives and composes the X-ray image which has been segmented and transmitted,
said X-ray imaging apparatus comprising a transmission unit configured to segment a captured X-ray image into images and sequentially transmit the images, and
said information processing apparatus comprising
a reception/composition unit configured to sequentially receive and compose the segmented and transmitted X-ray images,
a determination unit configured to determine, every time the X-ray images received by said reception/composition unit are sequentially composed, whether a resolution of the composed X-ray image is not less than a predetermined value, and
a display control unit configured to perform control to display the composed X-ray image on a display unit, when said determination unit determines that a resolution of the composed X-ray image is not less than the predetermined value.

2. The system according to claim 1, wherein said information processing apparatus further comprises an obtaining unit configured to obtain a resolution of the display unit, and
the predetermined value is the resolution of the display unit which is obtained by said obtaining unit.

3. The system according to claim 1, wherein said transmission unit comprises a region segmentation unit configured to segment the X-ray image into regions each including N pixels×N pixels,
a selection unit configured to select N pixels for said each region without overlapping, and
a pixel transmission unit configured to transmit N pixels selected for said each region, and
said selection unit selects N pixels different from the N pixels for said each region without overlapping when performing next transmission.

4. The system according to claim 3, wherein said selection unit selects the N pixels for said each region such that horizontal coordinates or vertical coordinates of the N pixels do not overlap on orthogonal coordinates with a predetermined pixel of the region being an origin point.

5. The system according to claim 4, wherein said selection unit selects, for said each region, N pixels arranged in a 45° direction or a −45° direction with respect to the orthogonal coordinates.

6. A method of controlling an X-ray imaging system comprising an X-ray imaging apparatus which captures an X-ray image and an information processing apparatus which receives and composes the X-ray image which has been segmented and transmitted, the method comprising:
segmenting, by a transmission unit of the X-ray imaging apparatus, a captured X-ray image into images and sequentially transmit the images;
receiving and composing, by a reception/composition unit of the information processing apparatus, sequentially the segmented and transmitted X-ray images;
determining, by a determination unit of the information processing apparatus, every time the X-ray images received in the receiving/composing are sequentially composed, whether a resolution of the composed X-ray image is not less than a predetermined value; and
performing, by a display control unit of the information processing apparatus, control to display the composed X-ray image on a display unit, when it is determined in the determining that a resolution of the composed X-ray image is not less than the predetermined value.

7. An information processing apparatus which receives and composes an X-ray image which has been segmented and sequentially transmitted from an X-ray imaging apparatus, the information processing apparatus comprising:
a reception/composition unit configured to sequentially receive and compose the segmented and transmitted X-ray images;
a determination unit configured to determine, every time the X-ray images received by said reception/composition unit are sequentially composed, whether a resolution of the composed X-ray image is not less than a predetermined value; and
a display control unit configured to perform control to display the composed X-ray image on a display unit, when said determination unit determines that a resolution of the composed X-ray image is not less than the predetermined value.

8. An X-ray imaging apparatus which captures an X-ray image, the apparatus comprising:
a region segmentation unit configured to segment the X-ray image into regions each including N pixels x N pixels;
a selection unit configured to select N pixels for said each region without overlapping; and
a pixel transmission unit configured to transmit N pixels selected for said each region,
wherein said selection unit selects N pixels different from the N pixels for said each region without overlapping when performing next transmission.
